(19) United States
(12) Patent Application Publication
(10) Pub. No.: US 2011/0243844 A1
(43) Pub. Date: Oct. 6, 2011

(54) SULFONAMIDE DERIVATIVE METABOTROPIC GLUTAMATE R4 LIGANDS

(76) Inventor: John A. McCauley, Rahway, NJ (US)

(21) Appl. No.: 12/737,942

(22) PCT Filed: Aug. 24, 2009
(86) PCT No.: PCT/US09/54763

§ 371 Date: May 13, 2011

Related U.S. Application Data
(60) Provisional application No. 61/192,140, filed on Sep. 16, 2008.

(51) Int. Cl. A61K 51/04; C07D 213/81; C07D 401/12; C07D 405/12; C07D 413/12; G01N 23/00

(52) U.S. Cl. 424/1.65; 546/323; 546/277.7; 546/282.4; 544/131; 546/153; 546/277.1; 546/141; 436/501

(57) ABSTRACT

Disclosed are mGluR4 positive allosteric modulator ligands of general formula (I) and radiolabeled derivates, their use as therapeutic agents for the treatment of central nervous system disorders modulated by mGluR4 and as ligands for the labeling and diagnostic imaging of mGluR4 in mammals.

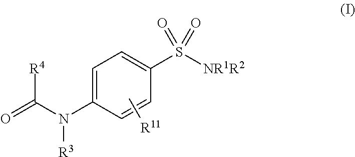

### FIELD OF THE INVENTION

The invention is directed to novel sulfonamide derivative ligands which are useful as metabotropic glutamate R4 (mGluR4) positive allosteric modulators, and to the use of the ligands as therapeutic compounds for treatment of central nervous system disorders modulated by mGluR4, and as imaging agents.

### BACKGROUND OF THE INVENTION

Glutamate is a key molecule in cellular metabolism, and is the most abundant excitatory neurotransmitter in the mammalian nervous system. Nerve impulses trigger release of glutamate from the pre-synaptic cell. In the opposing post-synaptic cell, glutamate receptors, such as the NMDA receptor, bind glutamate and are activated. Hence, glutamate mediates much of the excitatory neurotransmission within the mammalian central nervous system. Glutamate plays a role in a variety of physiological processes, such as long-term potentiation (learning and memory), the development of synaptic plasticity, motor control, respiration, cardiovascular regulation, and sensory perception. Hollmann et al, Annual Rev. Neurosci 17:31-108 (1994).

Glutamate acts via at least two distinct classes of receptors. One class is composed of the ionotropic glutamate (iGlu) receptors that act as ligand-gated ionic channels. Via activation of the iGlu receptors, glutamate is thought to regulate fast neuronal transmission within the synapse of two connecting neurons in the CNS. The second class of receptor is the G-protein or second messenger-linked “metabotropic” glutamate (mGluR) receptor. Both types of receptors appear not only to mediate normal synaptic transmission along excitatory pathways, but also participate in the modification of synaptic connections during development and throughout life. Schoepp et al, Trends in Pharmacol. Sci., 11, 508 (1990); McDonald et al, Brain Research Reviews, 15, 41 (1990).

The mGluR receptors belong to the Type III G-protein coupled receptor (GPCR) superfamily, which includes the calcium-sensing receptors, GABA_{B} receptors and pheromone receptors. The CPCR receptors are activated by binding of agonists to a large amino-terminus portion of the receptor protein. The mGluR receptors are thought to mediate glutamate's demonstrated ability to modulate intracellular signal transduction pathways. Ozawa et al, Prog. Neurobio., 54, 581 (1998). The mGluR receptors have been demonstrated to be localized both pre- and post-synaptically where they can regulate neurotransmitter release, or modify the post-synaptic response of neurotransmitters, respectively.

Diseases of the extrapyramidal motor systems cause either a loss of movement (akinesia) accompanied by an increase in muscle tone (rigidity) or abnormal involuntary movements (dyskinesias), often accompanied by a reduction in muscle tone. The akinetic-rigid syndrome called parkinsonism, and the dyskinesias represent opposite ends of the spectrum of movement disorders (C. D. Marsden in Oxford Textbook of Medicine, 3rd Edition, Oxford University Press, 1996, vol. 3, pages 3998-4022).

Glutamate mediates synaptic neurotransmission through the activation of inonotropic glutamate receptor channels, including the NMDS, AMPA and kainate receptors. Glutamate also activates metabotropic glutamate receptors (mGluR's) which have a modulatory role in synaptic efficacy.

It has been postulated that mGluR's play a role in a variety of pathophysiological processes and disease states affecting the CNS. These include stroke, head trauma, anoxic and ischemic injuries, addiction to cocaine, hypoglycemia, epilepsy, anxiety, schizophrenia and neurodegenerative diseases such as Alzheimer's disease. Schoepp et al (1993), Trends Pharmacol. Sci. 14:13; Cunningham et al. (1994), Life Sci. 54:135; Tatarczynska et al. (2001) Br. J. Pharmacol 132:1423-1430; Chiamulera et al. (2001) Nature Neurosci. 4:873-874; Chavez-Noriega et al. (2002) Current Drug Targets: CNS & Neurological Disorders 1:261-281. Much of the pathology in these conditions is thought to be due to excessive glutamate-induced excitation of CNS neurons.

It is believed that mGluR4 decreases GABAergic transmission with the basal ganglia, and thus may have a role in motor dysfunction. Reduction of gamma aminobutyric acid release by a selective agonist of mGluR4 has been suggested as an approach for the treatment of Parksinons' Disease. Marino et al, PNAS 100:13668-13673 (2003). Marino et al found that N-phenyl-7-(hydroxylimino)cycloprop-[b]chromen-1a-carboxamide (PHCC) acts as a potentiator of human and rat mGluR4.

### SUMMARY OF THE INVENTION

The present invention is directed to mGluR4 positive allosteric modulator ligands of general formula (I)

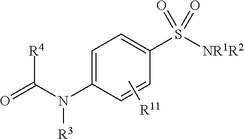

and radiolabeled derivates, and their use as therapeutic agents for the treatment of central nervous system disorders modulated by mGluR4. The invention is also directed to the use of radiolabeled mGluR4 ligands of the invention for the labeling and diagnostic imaging of mGluR4 in mammals. The invention is further directed to an assay for determining the mGluR4 binding properties of a test compound, using a radiolabeled mGluR4 ligand of the invention.

### DETAILED DESCRIPTION OF THE INVENTION

The invention is directed to sulfonamide derivative mGluR4 positive allosteric modulator ligands of formula (I)

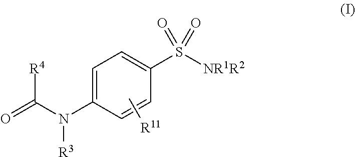

wherein:
R¹ and R² are each independently selected from the group consisting of
(1) hydrogen,
(2) —C₆₋₁₀ aryl,
(3) heteroaryl,
(4) —C₁₋₆ alkyl,
(5) —C₃₋₈ cycloalkyl,
wherein said R¹ or R² alkyl, cycloalkyl, aryl or heteroaryl moiety is optionally substituted with one or more
   (a) halogen,
   (b) —C₆₋₁₀ aryl,
   (c) heteroaryl,
   (d) —OC₁₋₄ alkyl,
   (e) —C₁₋₄ alkyl,
   (f) —CN,
   (g) —NO₂,
   (h) —C(═O)—R⁵,
   (i) —S(O)ₙR⁵,
   (j) —S(O)ₙ—NR⁹R¹⁰,
   (k) —S(O)ₙ—O—R⁵,
   (l) —C(═O)—OR⁵,
   (m) —NR⁹R¹⁰,
   wherein said alkyl or aryl moiety is optionally substituted with one or more
      (i) halogen,
      (ii) —CN,
      (iii) —OC₁₋₄ alkyl,
      (iv) —C₁₋₄ alkyl,
      (v) —C₆₋₁₀ aryl,
      (vi) heteroaryl,
         or R¹ and R² are linked together with the nitrogen to which they are both attached to form a monocyclic or bicyclic heterocyclic group, wherein said heterocyclic group is optionally substituted with one or more
(a) halogen,
(b) —C₁₋₄ alkyl, or
(c) —OC₁₋₄ alkyl, or
   provided that R¹ and R² are not both hydrogen;
   R³ is selected from the group consisting of
(1) hydrogen, or
(2) —C₁₋₄ alkyl;
   R⁴ is a heteroaryl group having at least one nitrogen atom, selected from the group consisting of pyridine, pyrimidine and quinolinimide, wherein said R⁴ heteroaryl is optionally substituted with one or more
(1) —C(═O)—OR⁵,
(2) —C₁₋₄ alkyl,
(3) —OC₁₋₄ alkyl, or
(4) halogen,
wherein said alkyl moiety is optionally substituted with one or more
   (a) halogen, or
   (b) hydroxyl;
      R⁵ is selected from the group consisting of
(1) hydrogen,
(2) —C₁₋₄ alkyl, or
(3) —C₆₋₁₀ aryl;
   R⁶, R⁷ and R⁸ are each independently selected from the group consisting of
(1) hydrogen, or
(2) —C₁₋₄ alkyl;
   R⁹ and R¹⁰ are each independently selected from the group consisting of
(1) hydrogen,
(2) —C₁₋₄ alkyl
(3) —C₆₋₁₀ aryl, or
(4) —C₃₋₈ cycloalkyl,
or R⁹ and R¹⁰ are linked together to form a heterocyclic group,
   R¹¹ is present at one or more of the phenyl ring carbon atoms, and each R¹¹ is independently selected from the group consisting of
(1) —C(═O)—OR⁵,
(2) —C₁₋₄ alkyl,
(3) —OC₁₋₄ alkyl, or
(4) halogen,
wherein said alkyl moiety is optionally substituted with one or more
   (a) halogen, or
   (b) hydroxyl;
      n is 0, 1 or 2;
      or a radiolabeled derivative thereof, and pharmaceutically acceptable salts thereof.

In one embodiment of the compounds of formula (I), R¹ is hydrogen or methyl and R² is selected from the group consisting of
(1) —C₆₋₁₀ aryl,
(2) heteroaryl,
(3) —C₁₋₆ alkyl,
(4) —C₃₋₈ cycloalkyl,
wherein said R² alkyl, cycloalkyl, aryl or heteroaryl moiety is optionally substituted with one or more
   (a) halogen,
   (b) —C₆₋₁₀ aryl,
   (c) heteroaryl,
   (d) —OC₁₋₄ alkyl,
   (e) —C₁₋₄ alkyl,
   (f) —CN,
   (g) —NO₂,
   (h) —C(═O)—R⁵,
   (i) —S(O)ₙR⁵,
   (j) —S(O)ₙ—NR⁹R¹⁰,
   (k) —S(O)ₙ—O—R⁵,
   (l) —C(═O)—OR⁵,
   (m) —NR⁹R¹⁰,
   wherein said alkyl or aryl moiety is optionally substituted with one or more
      (i) halogen,
      (ii) —CN,
      (iii) —OC₁₋₄ alkyl,
      (iv) —C₁₋₄ alkyl,
      (v) —C₆₋₁₀ aryl, or
      (vi) heteroaryl.

Typically, in this embodiment, R¹ is hydrogen or methyl (preferably hydrogen) and R² is selected from the group consisting of
(1) phenyl,
(2) pyridyl,
(3) pyrimidyl, or
(3) —C₁₋₆ alkyl, optionally substituted
wherein said R² is optionally substituted with one or more
   (a) halogen,
   (b) —OC₁₋₄ alkyl,
   (c) —C₁₋₄ alkyl,
   (d) phenyl,
   (d) —CN,
   (e) —C(═O)—R⁵,
   (f) —C(═O)—OR⁵, or
   (g) —NR⁹R¹⁰.

In other embodiments of the compounds of formula (I), R³ is hydrogen.

In one embodiment of the compounds of formula (I), R⁴ is pyridine.

In one embodiment, the compounds of formula (I) are compounds of formula (II):

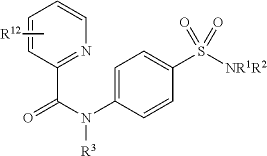

or a radiolabeled derivative thereof, and pharmaceutically acceptable salts thereof, wherein R¹, R² and R³ are as described above, and R¹² is present at one or more of the pyridine ring carbon atoms, and each R¹² is independently selected from the group consisting of
(1) —C(═O)—OR⁵,
(2) —C₁₋₄ alkyl,
(3) —OC₁₋₄ alkyl, or
(4) halogen,
wherein said alkyl moiety is optionally substituted with one or more
   (a) halogen, or
   (b) hydroxyl.

Typically, in this embodiment, R³ is hydrogen.

In another embodiment, the compounds of formula (I) are compounds of formula (III):

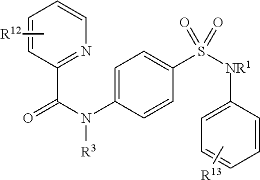

or a radiolabeled derivative thereof, and pharmaceutically acceptable salts thereof, wherein R¹ and R³ are as described above, R¹² is present at one or more of the pyridine ring carbon atoms, and each R¹³ is independently selected from the group consisting of
(1) —C₆₋₁₀ aryl,
(2) heteroaryl,
(3) —C₁₋₆ alkyl,
(4) —C₃₋₈ cycloalkyl,
wherein said R² alkyl, cycloalkyl, aryl or heteroaryl moiety is optionally substituted with one or more
   (a) halogen,
   (b) —C₆₋₁₀ aryl,
   (c) heteroaryl,
   (d) —OC₁₋₄ alkyl,
   (e) —C₁₋₄ alkyl,
   (f) —CN,
   (g) —NO₂,
   (h) —C(═O)—R⁵,
   (i) —S(O)ₙR⁵,
   (j) —S(O)ₙ—NR⁹R¹⁰,
   (k) —S(O)ₙ—O—R⁵,
   (l) —C(═O)—OR⁵,
   (m) —NR⁹R¹⁰,
   wherein said alkyl or aryl moiety is optionally substituted with one or more
      (i) halogen,
      (ii) —CN,
      (iii) —OC₁₋₄ alkyl,
      (iv) —C₁₋₄ alkyl,
      (v) —C₆₋₁₀ aryl, or
      (vi) heteroaryl.

Typically, in this embodiment, R³ is hydrogen.

In particular embodiments, the invention is directed to radiolabeled compounds of formula (I), for example tritium labeled compounds.

In another embodiment, the invention is directed to any of exemplary compounds 1-50, including, for example:
N-(4-{[(2-Chlorophenyl)amino]sulfonyl}phenyl)pyridine-2-carboxamide (Example 1);
N-(4-{[(2-Chloro-4-iodophenyl)amino]sulfonyl}phenyl)-4-iodopyridine-2-carboxamide (Example 2);
[³H]-N-(4-{[(2-Chlorophenyl)amino]sulfonyl}phenyl)pyridine-2-carboxamide (Example 3);
N-(4-{[(2-benzoylphenyl)amino]sulfonyl}phenyl)pyridine-2-carboxamide (Example 4);
   or a radiolabeled deriviative, or a pharmaceutically acceptable salt thereof.

The invention is also directed to an assay for determining the binding affinity of a test compound to the mGluR4 receptor, comprising the steps of
(1) preparing a membrane from a cell expressing the human mGluR4 receptor;
(2) forming a solution comprising
   (a) the membrane,
   (b) a radiolabeled compound of formula (I),
   (c) a test compound, and
   (d) an mGluR4 orthosteric agonist;
(3) incubating the solution;
(4) collecting the membrane from the solution;
(5) determining the amount of radioactivity bound to the mGluR4 receptor; and
(6) calculating the affinity of the test compound for the mGluR4 receptor.

In one embodiment, the compound of formula (I) is radiolabeled with tritium.

In one embodiment, the mGluR4 orthosteric agonist is L-AP4 ((S)-2-amino-4-phosphonobutanoate.

In one embodiment of the assay, the compound of formula (I) is N-(4-{[(2-Chlorophenyl)amino]sulfonyl}phenyl)pyridine-2-carboxamide, radiolabeled with tritium, for example [³H]-N-(4-{[(2-Chlorophenyl)amino]sulfonyl}-phenyl)pyridine-2-carboxamide

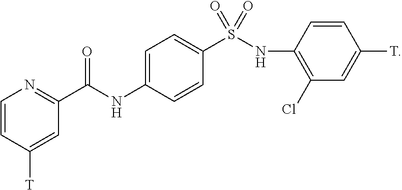

The compounds of the present invention may contain one or more chiral centers and can thus occur as racemates and racemic mixtures, single enantiomers, diastereomeric mixtures and individual diastereomers. Additional asymmetric centers may be present depending upon the nature of the various substituents on the molecule. Each such asymmetric center will independently produce two optical isomers, and it is intended that all of the possible optical isomers and diastereomers in mixtures and as pure or partially purified compounds are included within the scope of this invention. The present invention includes all such isomeric forms of these compounds.

The independent syntheses of these diastereomers or their chromatographic separations may be achieved as known in the art by appropriate modification of the methodology disclosed herein. Their absolute stereochemistry may be determined by the x-ray crystallography of crystalline products or crystalline intermediates which are derivatized, if necessary, with a reagent containing an asymmetric center of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

Alternatively, any enantiomer of a compound may be obtained by stereoselective synthesis using optically pure starting materials or reagents of known configuration by methods well known in the art.

The term “halo” or “halogen” as used herein includes fluoro, chloro, bromo and iodo. Similarly, C₁₋₃, as in C₁₋₃ alkyl is defined to identify the group as having 1, 2 or 3 carbons in a linear or branched arrangement, such that C₁₋₃alkyl specifically includes methyl, ethyl, n-propyl, and iso-propyl. A group which is designated as being independently substituted with substituents may be independently substituted with multiple numbers of such substituents.

As used herein, the term “alkyl” means linear or branched structures having no carbon-to-carbon double or triple bonds. Thus C₁₋₆ alkyl is defined to identify the group as having 1, 2, 3, 4, 5 or 6 carbons in a linear or branched arrangement, such that C₁₋₆ alkyl specifically includes, but is not limited to, methyl, ethyl, n-propyl, iso-propyl, n-butyl, iso-butyl, tert-butyl, pentyl and hexyl. “Cycloalkyl” is an alkyl, part or all of which forms a ring of three or more atoms. C₀ or C₀alkyl is defined to identify the presence of a direct covalent bond.

As used herein, the term “aryl” is intended to mean any stable monocyclic or bicyclic carbon ring of up to 7 members in each ring, wherein at least one ring is aromatic. Examples of such aryl groups include phenyl, napthyl, tetrahydronapthyl, indanyl, or biphenyl.

As used herein, the term “heteroaryl,” by itself or as part of another substituent, means an aromatic cyclic group having at least one ring heteroatom (O, N or S). The term “heteroaryl” includes multiple ring systems as well as single ring systems, and includes multiple ring systems wherein part of the molecule is aromatic and part is non-aromatic. Preferred heteroaryl groups have from 5 to 12 ring atoms. Exemplary heteroaryl groups include pyrazinyl, pyrazolyl, pyridazinyl, pyridyl, pyrimidinyl, pyrrolyl, tetrazolyl, furanyl, imidazolyl, indazolyl, triazinyl, pyranyl, thiazolyl, thienyl, thiophenyl, triazolyl, oxazolyl, isoxazolyl, thiazolyl, oxadiazolyl, indolyl, quinolinyl, isoquinolinyl, benzimidazolyl, benzofuranyl and benzoxazolyl. More preferred heteroaryl groups include indolyl, thienyl, pyridinyl, dihydroquinolinyl and tetrahydroquinolinyl.

When a heteroaryl group as defined herein is substituted, the substituent may be bonded to a ring carbon atom of the heteroaryl group, or on a ring heteroatom (i.e., a nitrogen, oxygen or sulfur), which has a valence which permits substitution. Preferably, the substituent is bonded to a ring carbon atom. Similarly, when a heteroaryl group is defined as a substituent herein, the point of attachment may be at a ring carbon atom of the heteroaryl group, or on a ring heteroatom (i.e., a nitrogen, oxygen or sulfur), which has a valence which permits attachment. Preferably, the attachment is at a ring carbon atom.

The term “pharmaceutically acceptable” means that the carrier, diluent or excipient must be compatible with the other ingredients of the formulation and not deleterious to the recipient thereof.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts in the solid form may exist in more than one crystal structure, and may also be in the form of hydrates. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylene-diamine, diethylamine, 2-diethylaminoethanol, 2-dimethylamino-ethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, fumaric, and tartaric acids. It will be understood that, as used herein, references to the compounds of the present invention are meant to also include the pharmaceutically acceptable salts.

The term “composition” as used herein is intended to encompass a product comprising the specified ingredients in the specified amounts, as well as any product which results, directly or indirectly, from combination of the specified ingredients in the specified amounts.

The terms “administration of” and or “administering a” mean providing a compound of the invention or a prodrug of a compound of the invention to the patient.

### Therapeutic Uses

In a first embodiment, the invention is directed to methods of treating a patient (preferably a human) for diseases or disorders modulated by mGluR4, such as Parkinson's Disease, by administering to the patient a therapeutically effective amount of a compound of general formula (I), or a pharmaceutically acceptable salt thereof.

The invention is also directed to the use of a compound of formula (I), or a pharmaceutically acceptable salt thereof, for treating diseases or disorders modulated by mGluR4, such as Parkinson's Disease.

The invention is also directed to medicaments or pharmaceutical compositions for treating diseases or disorders modulated by mGluR4, such as Parkinson's Disease, which comprise a compound of formula (I), or a pharmaceutically acceptable salt thereof, and a pharmaceutically acceptable carrier.

The invention is further directed to a method for the manufacture of a medicament or a composition for treating diseases or disorders modulated by mGluR4, such as Parkinson's Disease, by combining a compound of formula (I), or a pharmaceutically acceptable salt thereof, with one or more pharmaceutically acceptable carriers.

In its embodiment as a therapeutic, the compounds of the invention are typically administered as pharmaceutical compositions. The pharmaceutical compositions may be used in the form of a pharmaceutical preparation, for example, in solid, semisolid or liquid form, which contains one or more of the compound of the present invention, as an active ingredient, in admixture with an organic or inorganic carrier or excipient suitable for external, enteral or parenteral applications. The active ingredient may be compounded, for example, with the usual non-toxic, pharmaceutically acceptable carriers for tablets, pellets, capsules, suppositories, solutions, emulsions, suspensions, and any other form suitable for use. The carriers which can be used are water, glucose, lactose, gum acacia, gelatin, mannitol, starch paste, magnesium trisilicate, talc, corn starch, keratin, colloidal silica, potato starch, urea and other carriers suitable for use in manufacturing preparations, in solid, semisolid, or liquid form, and in addition auxiliary, stabilizing, thickening and coloring agents and perfumes may be used. The active object compound is included in the pharmaceutical composition in an amount sufficient to produce the desired effect upon the process or condition of the disease.

The liquid forms in which the novel compositions of the present invention may be incorporated for administration orally or by injection include aqueous solution, suitably flavoured syrups, aqueous or oil suspensions, and emulsions with acceptable oils such as cottonseed oil, sesame oil, coconut oil or peanut oil, or with a solubilizing or emulsifying agent suitable for intravenous use, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinylpyrrolidone or gelatin.

An appropriate dosage level for the use of the mGluR4 positive allosteric modulators of the invention as therapeutic agents will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 milligrams of the active ingredient, particularly 1.0, 5.0, 10, 15, 20, 25, 50, 75, 100, 150, 200, 250, 300, 400, 500, 600, 750, 800, 900, and 1000 milligrams of the active ingredient for the symptomatic adjustment of the dosage to the patient to be treated. The compounds may be administered on a regimen of 1 to 4 times per day, preferably once or twice per day. This dosage regimen may be adjusted to provide the optimal therapeutic response. It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Exemplary diseases modulated by mGluR4, for which the compounds of the invention may be useful, are central nervous system disorders, such as addiction, tolerance or dependence; affective disorders, such as depression and anxiety; psychiatric diseases such as psychotic disorders, attention-deficit/hyperactivity disorder and bipolar disorder; Parkinson's disease; memory impairment; Alzheimer's disease; dementia; delirium tremens; other forms of neurodegeneration, neurotoxicity and ischemia.

In particular, the compounds of the invention may be useful as therapeutic agents for treating or preventing Parkinson's disease, and movement disorders such as bradykinesia, rigidity, dystonia, drug-induced parkinsonism, dyskinesia, tardive dyskinesia, L-DOPA-induced dyskinesia, dopamine agonist-induced dyskinesia, hyperkinetic movement disorders, Gilles de la Tourette syndrome, resting tremor, action tremor, akinesia, akinetic-rigid syndrome, akathisia, athetosis, asterixis, tics, postural instability, postencephalitic parkinsonism, muscle rigidity, chorea and choreaform movements, spasticity, myoclonus, hemiballismus, progressive supranuclear palsy, restless legs syndrome and periodic limb movement disorder.

In another embodiment, the compounds of the invention may be useful for treating or preventing cognitive disorders, such as delirium, substance-induced persisting delirium, dementia, dementia due to HIV disease, dementia due to Huntington's disease, dementia due to Parkinson's disease, Parkinsonian-ALS demential complex, dementia of the Alzheimer's type, substance-induced persisting dementia and mild cognitive impairment.

In another embodiment, the compounds of the invention may be useful for treating or preventing affective disorders, anxiety, agoraphobia, generalized anxiety disorder (GAD), obsessive-compulsive disorder (OCD), panic disorder, posttraumatic stress disorder (PTSD), social phobia, other phobias, substance-induced anxiety disorder, acute stress disorder, mood disorders, bipolar disorders (I & II), cyclothymic disorder, depression, dysthymic disorder, major depressive disorder, substance-induced mood disorder; multiple sclerosis, including benign multiple sclerosis, relapsing-remitting multiple sclerosis, secondary progressive multiple sclerosis, primary progressive multiple sclerosis and progressive-relapsing multiple sclerosis.

In another embodiment, the compounds of the invention may be useful for treating or preventing epilepsy and tremor, temporal lobe epilepsy, epilepsy secondary to another disease or injury e.g., chronic encephalitis.

In another embodiment, the compounds of the invention may be useful for treating or preventing traumatic brain injury, stroke, ischemia, spinal cord injury, cerebral hypoxia or intracranial hematoma.

In another embodiment, the compounds of the invention may be useful for treating or preventing medulloblastomas.

In another embodiment, the compounds of the invention may be useful for treating or preventing inflammatory or neuropathic pain.

In another embodiment, the compounds of the invention may be useful for treating or preventing various metabolic disorders associated with glutamate dysfunction.

In another embodiment, the compounds of the invention may be useful for treating or preventing type 2 diabetes.

In another embodiment, the compounds of the invention may be useful for treating or preventing diseases or disorders of the retina, retinal degeneration or macular degeneration.

In another embodiment, the compounds of the invention may be useful for treating or preventing diseases or disorders of the gastrointestinal tract, including gastro-esophageal reflux disease (GERD), lower esophageal sphincter diseases or disorders, diseases of gastrointestinal motility, colitis, Crohn's disease or irritable bowel syndrome (IBS).

### Imaging Uses

In another embodiment, the compounds of the invention may be labeled as radionuclides, for use in imaging. For example, the compounds may be prepared as Positron Emission Tomography (PET) radiotracers, for use in imaging for clinical evaluation and dose selection of mGluR4 positive allosteric modulator or other mGluR4 ligand. Using a fluorine-18 or carbon-11 labeled radiotracer that provides a mGluR4-specific image in the brain and other tissues, the dose required to saturate the mGluR4 receptor can be determined by the blockade of the PET radiotracer image in humans.

Suitable radionuclides that may be incorporated in the instant compounds include ²H or deuterium (also written as D), ³H or tritium (also written as T), 11C, ¹⁸F, ¹²⁵I, ⁸²Br, ¹²³I, ¹³¹I, ⁷⁵Br, ¹⁵O, ¹³N, ²¹¹At or ⁷⁷Br. The radionuclide that is incorporated in the instant radiolabeled compounds will depend on the specific analytical or pharmaceutical application of that radiolabeled compound. Thus, for in vitro labeling of mGluR4 and competition assays, compounds that incorporate ³H, ¹²⁵I or ⁸²Br will generally be most useful. For diagnostic imaging agents, compounds that incorporate a radionuclide selected from ¹¹C, ¹⁸F, ¹²³I, ¹³¹I, ⁷⁵Br, ⁷⁶Br or ⁷⁷Br are preferred. In certain applications incorporation of a chelating radionuclide such as Tc^{99m} may also be useful. ¹⁸F may be preferable over ¹¹C because with the longer half-life of ¹⁸F, imaging can be carried out long enough to allow a more specific signal to develop and improved conditions for receptor quantification studies.

In this embodiment, the compounds of the invention can be labeled with either positron or gamma emitting radionuclides. For imaging, the most commonly used positron emitting (PET) radionuclides are ¹¹C, ¹⁸F, ¹⁵O and ¹³N, all of which are accelerator produced, and have half lifes of 20, 110, 2 and 10 minutes, respectively. Since the half-lives of these radionuclides are so short, it is only feasible to use them at institutions that have an accelerator on site or very close by for their production, thus limiting their use. Several gamma emitting radiotracers are available which can be used by essentially any hospital in the U.S. and in most hospitals worldwide. The most widely used of these are ^{99m}Tc, ²⁰¹Tl and ¹²³I.

The radiolabeled mGluR4 positive allosteric modulators of the present invention have utility in imaging mGluR4 or for diagnostic imaging with respect to any of the previously mentioned neurological and psychiatric disorders associated with mGluR4 neurotransmission dysfunction

The present invention is also directed to a method for diagnostic imaging of mGluR4 in a mammal which comprises administering to a mammal in need of such diagnostic imaging an effective amount of the radiolabeled compound of the present invention.

The present invention is also directed to a method for diagnostic imaging of tissues bearing mGluR4 in a mammal which comprises administering to a mammal in need of such diagnostic imaging an effective amount of the radiolabeled compound of the present invention.

The present invention is also directed to a method for the diagnostic imaging of mGluR4 in tissues of a mammalian species which comprises administering to the mammalian species in need of such diagnostic imaging an effective amount of the radiolabeled compound of the present invention.

The present invention is also directed to a method for diagnostic imaging of the brain in a mammal which comprises administering to a mammal in need of such diagnostic imaging an effective amount of the radiolabeled compound of the present invention.

The present invention is further directed to a method for the detection or quantification of mGluR4 in mammalian tissue which comprises administering to a mammal in which such quantification is desired an effective amount of the radiolabeled compound of the present invention.

In a preferred embodiment of the methods of the present invention, the mammal is a human.

The invention is also directed to an mGluR4 binding assay, using a radiolabeled compound of the invention.

In one embodiment of the assay, membranes are prepared from cells expressing the human mGluR4 receptor. Typically, CHO cells are used. The cells are harvested and washed in an assay buffer, according to methods know to those skilled in the art. Typical assay buffers include HEPES, EDTA and protease inhibitors. The cells may then be pelleted, stored and resuspended according to standard assay procedures.

The binding assay of the invention may follow a standard filtration binding paradigm, known to those skilled in the art. A binding buffer is prepared. Typical binding buffers comprise HEPES, NaCl and MgCl₂. A suitable pH for the buffer is between 7 and 8, for example about 7.4.

The assay may be performed in a multi-well format (for example, a 96-well format) with each well containing test compound, about 25 to 50 ug of membrane protein, about 20 uM of an orthosteric mGluR4 agonist, such as L-AP4, and about 7 nM of a radiotracer, which is a radiolabeled compound of formula (I). For the radiolabeled mGluR4 potentiators examined, specific binding to the mGluR4 receptor requires the presence of an orthosteric agonist, such as L-AP4. The binding reaction is incubated at room temperature, for example for one hour, and then is passed through a filter, typically presoaked with a PEI solution, to collect the cell membranes. Typically, the membrane is then washed with a buffer. The cell membranes are collected and washed, and the filter plate may then be dried. A scintillation fluid may be added, and the amount of filter bound radioactivity is determined (for example, using a TopCount instrument). Total binding may be determined in the absence of a test compound, but in the presence of the DMSO concentration that would result from the addition of test compound. To determine the level of non-specific binding for the radiotracer, an amount of the unlabeled compound is added to determine the level of non-specific binding. The inflection point of the curve, or IC₅₀, is calculated to provide a measure of the affinity of a test compound for the mGluR4 receptor.

Radiolabeled mGluR4 positive allosteric modulators, when labeled with the appropriate radionuclide, are potentially useful for diagnostic imaging, basic research, and radiotherapeutic applications. Specific examples of possible diagnostic imaging and radiotherapeutic applications, include determining the location, the relative activity and/or the abundance of mGluR4, radioimmunoassay of mGluR4 positive allosteric modulators, and autoradiography to determine the distribution of mGluR4 positive allosteric modulators in a mammal or an organ or tissue sample thereof.

In particular, the instant radiolabeled mGluR4 positive allosteric modulators when labeled with a positron emitting radionuclide, are useful for positron emission tomographic (PET) imaging of mGluR4 in the brain of living humans and experimental animals. These radiolabeled mGluR4 positive allosteric modulators may be used as research tools to study the interaction of unlabeled mGluR4 positive allosteric modulators with mGluR4 in vivo via competition between the labeled drug and the radiolabeled compound for binding to the receptor. These types of studies are useful for determining the relationship between mGluR4 occupancy and dose of unlabeled mGluR4 positive allosteric modulators, as well as for studying the duration of blockade of the receptor by various doses of the unlabeled mGluR4 positive allosteric modulators. As a clinical tool, the radiolabeled mGluR4 positive allosteric modulators may be used to help define a clinically efficacious dose of an mGluR4 positive allosteric modulator. In animal experiments, the radiolabeled mGluR4 positive allosteric modulators can be used to provide information that is useful for choosing between potential drug candidate for selection for clinical development. The radiolabeled mGluR4 positive allosteric modulators may also be used to study the regional distribution and concentration of mGluR4 in the living human brain, as well as the brain of living experimental animals and in tissue samples. The radiolabeled mGluR4 positive allosteric modulators may also be used to study disease or pharmacologically related changes in mGluR4 concentrations.

For example, positron emission tomography (PET) tracers such as the present radiolabeled mGluR4 positive allosteric modulators which can be used with currently available PET technology to obtain the following information: relationship between level of receptor occupancy by candidate mGluR4 positive allosteric modulators and clinical efficacy in patients; dose selection for clinical trials of mGluR4 positive allosteric modulators prior to initiation of long term clinical studies; comparative potencies of structurally novel mGluR4 positive allosteric modulators; investigating the influence of mGluR4 positive allosteric modulators on in vivo transporter affinity and density during the treatment of clinical targets with mGluR4 positive allosteric modulators and other agents; changes in the density and distribution of mGluR4 positive allosteric modulators during e.g. psychiatric diseases in their active stages, during effective and ineffective treatment and during remission; and changes in mGluR4 expression and distribution in CNS disorders; imaging neurodegenerative disease where mGluR4 is upregulated; imaging neurodegenerative disease where mGluR4 is involved; and the like.

For the use of the instant compounds as exploratory or diagnostic imaging agents the radiolabeled compounds may be administered to mammals, preferably humans, in a pharmaceutical composition, either alone or, preferably, in combination with pharmaceutically acceptable carriers or diluents, optionally with known adjuvants, such as alum, in a pharmaceutical composition, according to standard pharmaceutical practice. Such compositions can be administered orally or parenterally, including the intravenous, intramuscular, intraperitoneal, subcutaneous, rectal and topical routes of administration. Preferably, administration is intravenous. Radiotracers labeled with short-lived, positron emitting radionuclides are generally administered via intravenous injection within less than one hour of their synthesis. This is necessary because of the short half-life of the radionuclides involved (20 and 110 minutes for C-11 and F-18 respectively).

When a radiolabeled mGluR4 positive allosteric modulator according to this invention is administered into a human subject, the amount required for diagnostic imaging will normally be determined by the prescribing physician with the dosage generally varying according to the age, weight, and response of the individual patient, as well as the quantity of emission from the radionuclide. However, in most instances, an effective amount will be the amount of compound sufficient to produce emissions in the range of from about 1-5mCi.

In one exemplary application, administration occurs in an amount of radiolabeled compound of between about 0.005 μg/kg of body weight to about 50 μg/kg of body weight per day, preferably of between 0.02 μg/kg of body weight to about 3 μg/kg of body weight. A particular analytical dosage that comprises the instant composition includes from about 0.5 μg to about 100 μg of a labeled mGluR4 positive allosteric modulator. Preferably, the dosage comprises from about 1 μg to about 50 μg of a radiolabeled mGluR4 positive allosteric modulator.

The following illustrative procedure may be used when performing PET imaging studies on patients in the clinic. The patient is premedicated with unlabeled mGluR4 positive allosteric modulator (at doses 300, 100, or 30 mg/day) for 2 weeks prior to the day of the experiment and is fasted for at least 12 hours allowing water intake ad libitum. A 20 G two inch venous catheter is inserted into the contralateral ulnar vein for radiotracer administration.

The patient is positioned in the PET camera and a tracer dose of [¹⁵O]H₂O administered via i.v. catheter. The image thus obtained is used to insure that the patient is positioned correctly to include the brain or other areas of interest. Subsequently the radiolabled mGluR4 positive allosteric modulator (<20 mCi) is administered via i.v. catheter. Following the acquisition of the total radiotracer image, an infusion is begun of the mGluR4 positive allosteric modulator which is being clinically evaluated at one of three dose rates (0.1, 1 or 10 mpk/day). After infusion for 2.5 hrs, the radiolabeled mGluR4 positive allosteric modulator is again injected via the catheter. Images are again acquired for up to 90 min. Within ten minutes of the injection of radiotracer and at the end of the imaging session, 1 ml blood samples are obtained for determining the plasma concentration of the clinical candidate.

For determining the distribution of radiotracer, regions of interest (ROIs) are drawn on the reconstructed image including, e.g. the brain and the central nervous system. These regions are used to generate time activity curves obtained in the absence of receptor antagonist or in the presence of the clinical candidate at the various infusion doses examined. Data are expressed as radioactivity per unit time per unit volume (μCi/cc/mCi injected dose). Inhibition curves are generated from the data obtained in a region of interest obtained starting at 70 minutes post-injection of radiotracer. At this time, clearance of non-specific binding has reached steady state. The ID₅₀ values, the dose of compound which inhibits 50% of specific radiotracer binding to mGluR4, may then be calculated.

Several methods for preparing the compounds of this invention are illustrated in the following Schemes and Examples. Starting materials and the requisite intermediates are in some cases commercially available, or can be prepared according to literature procedures or as illustrated herein.

The compounds of this invention may be prepared by employing reactions as shown in the following schemes, in addition to other standard manipulations that are known in the literature or exemplified in the experimental procedures. Substituent numbering as shown in the schemes does not necessarily correlate to that used in the claims and often, for clarity, a single substituent is shown attached to the compound where multiple substituents are allowed under the definitions hereinabove. Reactions used to generate the compounds of this invention are prepared by employing reactions as shown in the schemes and examples herein, in addition to other standard manipulations such as ester hydrolysis, cleavage of protecting groups, etc., as may be known in the literature or exemplified in the experimental procedures.

In some cases the final product may be further modified, for example, by manipulation of substituents. These manipulations may include, but are not limited to, reduction, oxidation, alkylation, acylation, and hydrolysis reactions which are commonly known to those skilled in the art. In some cases the order of carrying out the foregoing reaction schemes may be varied to facilitate the reaction or to avoid unwanted reaction products. The following examples are provided so that the invention might be more fully understood. These examples are illustrative only and should not be construed as limiting the invention in any way.

The compounds of the present invention may be synthesized as outlined below. An appropriately substituted aniline can be reacted with various acid chlorides in a solvent with an inorganic or organic base (for example, triethylamine) as an acid scavenger. In the second step, a sulfonyl chloride group can be introduced through reaction with chlorosulfonic acid, and then resulting sulfonamides can be prepared though reaction with appropriately substituted amines in the presence of an inorganic or organic base.

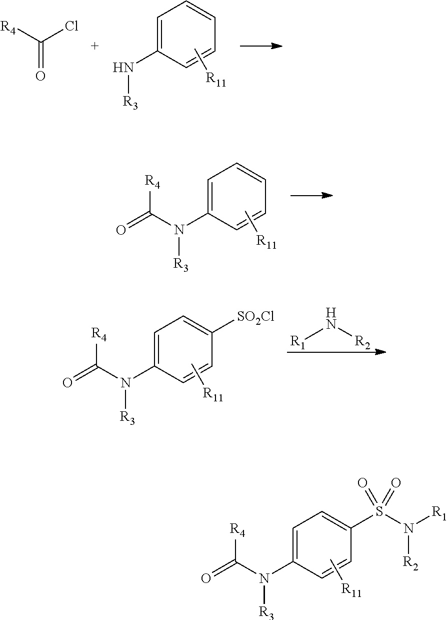

Syntheses of particular mGluR4 positive allosteric modulators are described below. During any of the above synthetic sequences it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene and P. G. M. Wuts, Protective Groups in Organic Synthesis, John Wiley & Sons, 1991. The protecting groups may be removed at a convenient subsequent stage using methods known from the art. In particular, amino moieties may be protected by, for example, the formation of alkoxycarbonyl derivatives, e.g. tert-butoxycarbonyl and trichloroethoxycarbonyl, or benzyl, trityl or benzyloxycarbonyl derivatives. Subsequent removal of the protecting group is achieved by conventional procedures thus, for example, tert-butoxycarbonyl, benzyl or benzyloxycarbonyl groups may be removed by hydrogenolysis in the presence of a catalyst e.g. palladium; a trichloroethoxycarbonyl group may be removed with zinc dust; and a trityl group may be removed under acidic conditions using standard procedures. Where hydroxyl groups require protection, this may be effected by the formation of esters or trialkylsilyl, tetrahydropyran or benzyl ethers. Such derivatives may be deprotected by standard procedures thus, for example, a tetrahydropyran ether derivative may be deprotected using hydrochloric acid in methanol.

mGluR4 positive allosteric modulators which incorporate a radionuclide may be prepared by first synthesizing an unlabeled compound that optionally incorporates a iodo or bromo moiety and then exchanging a hydrogen or halogen moiety with an appropriate radionuclide using techniques well known in the art. Alternately, a radiolabeled mGluR4 positive allosteric modulator may be prepared by alkylation with a radiolabeled alkylating agent.

### Example 1

### N-(4-{[(2-Chlorophenyl)amino]sulfonyl}phenyl)pyridine-2-carboxamide

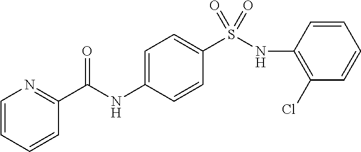

### Step 1: N-(2-Chlorophenyl)-4-nitrobenzenesulfonamide

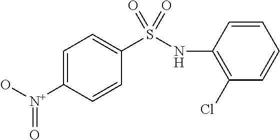

To a solution of 2-chloroaniline (3.45 g, 27.0 mmol) in pyridine (50 mL) at RT was added p-nitrobenzenesulfonyl chloride (5.0 g, 22.6 mmol) and the reaction mixture was stirred at RT for 18 h. The reaction mixture was diluted with 10% KHSO₄ (aq) and EtOAc and the layers were separated. The organic layer was washed with 10% NaHCO₃ (aq), dried over Na₂SO₄, filtered and concentrated. The crude product was purified by silica gel chromatography (gradient elution, 10% to 50% EtOAc in hexanes) followed by trituration in 2% EtOAc in hexane and filtration to give the title product.

### Step 2: 4-Amino-N-(2-chlorophenyl)benzenesulfonamide

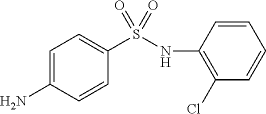

To a solution of N-(2-chlorophenyl)-4-nitrobenzenesulfonamide (4.0 g, 12.8 mmol) in 80% EtOH/EtOAc (500 mL) was added Raney Ni (1 g). The reaction mixture was stirred under a 1 atm H₂ atmosphere for 6 h, filtered and concentrated to give the title product. LCMS (ESI) m/z 324.2 [(M+MeCN+H)⁺; calcd for C₁₂H₁₂ClN₂O₂S—CH₃CN: 324.1].

### Step 3: N-(4-{[(2-Chlorophenyl)amino]sulfonyl}phenyl)pyridine-2-carboxamide

To a solution of 4-amino-N-(2-chlorophenyl)benzenesulfonamide (3.0 g, 10.6 mmol) in THF (60 mL) at RT was added TEA (5.93 mL, 42.6 mL) and pyridine-2-carbonyl chloride (2.84 g, 16.0 mmol). The reaction mixture was stirred at RT for 4 h, was diluted with EtOAc and 10% NaHCO3 (aq) and stirred for 30 min. The layers were separated and the aqueous layer was back-extracted with EtOAc. The combined organic layers were dried over Na₂SO₄, filtered and concentrated. The crude product was purified by silica gel chromatography (gradient elution, 20% to 60% EtOAc in hexanes). The product was crystallized from EtOAc and filtered to give the title compound as a white solid. ¹H NMR (400 MHz, CDCl₃) δ 10.24 (s, 1H), 8.61 (d, J=4.6 Hz, 1H), 8.28 (d, J=7.9 Hz, 1H), 7.93 (dt, J=7.7, 1.5 Hz, 1H), 7.85 (d, J=8.9 Hz, 2H), 7.77 (d, J=8.9 Hz, 2H), 7.68 (dd, J=8.2, 1.1 Hz, 1H), 7.51 (m, 1H), 7.25-7.22 (m, 2H), 7.04 (dt, J=8.2, 1.4 Hz, 1H), 6.98 (s, 1H) ppm; LCMS (ESI) m/z 388.2 [(M+H)⁺; calcd for C₁₈H₁₅ClN₃O₃S: 388.1].

### Example 2

### N-(4-{[(2-Chloro-4-iodophenyl)amino]sulfonyl}phenyl)-4-iodopyridine-2-carboxamide

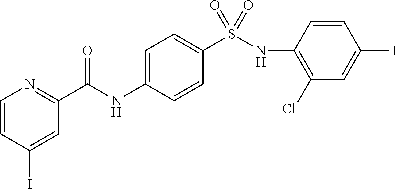

### Step 1: N-(2-Chloro-4-iodophenyl)-4-nitrobenzenesulfonamide

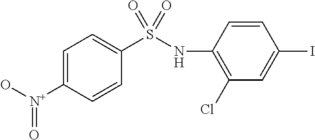

To a solution of 2-chloro-4-iodoaniline (1.26 g, 5.0 mmol) in pyridine (10 mL) at RT was added p-nitrobenzenesulfonyl chloride (1.0 g, 4.5 mmol) and the reaction mixture was stirred at RT for 18 h. The reaction mixture was diluted with 10% KHSO₄ (aq) and EtOAc and the layers were separated. The organic layer was washed with 10% NaHCO₃ (aq), dried over Na₂SO₄, filtered and concentrated. The crude product was purified by silica gel chromatography (DCM to 2% acetone in DCM) gave the title product.

### Step 2: 4-Amino-N-(2-chloro-4-iodophenyl)benzenesulfonamide

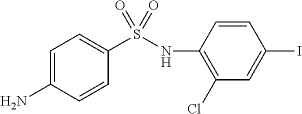

To a solution of N-(2-chloro-4-iodophenyl)-4-nitrobenzenesulfonamide (0.85 g) in 90% EtOH/EtOAc (200 mL) was added Raney Ni (1 g). The reaction mixture was stirred under a 1 atm H₂ atmosphere for 3 h, filtered and concentrated to give the title product which contains approximately 15% of the des-iodo product.

### Step 3: N-(4-{[(2-Chloro-4-iodophenyl)amino]sulfonyl}phenyl)-4-iodopyridine-2-carboxamide

To a solution of 4-amino-N-(2-chloro-4-iodophenyl)benzenesulfonamide (224 mg, 0.55 mmol) and 4-iodopyridine-2-carboxylic acid (207 mg, 0.55 mmol) in DMF (5 mL) was added EDC (157 mg, 0.82 mmol), HOAt (126 mg, 0.82 mmol and TEA (0.19 mL, 1.37 mmol). The reaction mixture was stirred at 70 C. for 4 h, cooled and purified by preparative reverse-phase HPLC to remove the mono-iodo product. The desired compound was chromatographed on silica gel (gradient elution, DCM to 5% acetone in DCM) to give the title compound. ¹H NMR (400 MHz, CDCl₃) δ 10.11 (s, 1H), 8.65 (d, J=1.6 Hz, 1H), 8.25 (d, J=5.1 Hz, 1H), 7.90 (dd, J=5.1, 1.7 Hz, 1H), 7.85 (d, J=8.8 Hz, 2H), 7.78 (d, J=8.8 Hz, 2H), 7.60-7.52 (m, 2H), 7.41 (d, J=8.5 Hz, 1H), 6.92 (s, 1H) ppm; LCMS (ESI) m/z 639.9 [(M+H)⁺; calcd for C₁₈H₁₃ClI₂N₃O₃S: 639.8].

### Example 3

### [³H]-N-(4-{[(2-Chlorophenyl)amino]sulfonyl}phenyl)pyridine-2-carboxamide

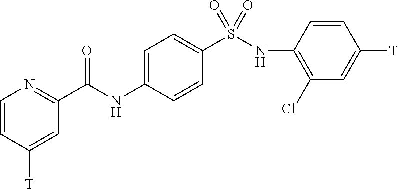

Di-iodo-precursor (N-(4-{[(2-Chloro-4-iodophenyl)amino]sulfonyl}phenyl)-4-iodopyridine-2-carboxamide, Example 5, 3 mg, 3.9×10⁻³ mmol) was dissolved in DMF (800 pit) and 5% Pd/C (Aldrich, 10 mg) and 5% Pd/CaCO₃ (Aldrich, 5 mg) stirred at room temperature in a 2 mL reaction flask attached to a INUS Trisorber Tritiation Manifold gas outlet. The content was frozen in liquid nitrogen and de-gassed to 4 mmHg. Carrier-free tritium gas (ARC, 2.5 mL at 740 mmHg. 5Ci, 2 Ci/mL) was introduced from the manifold and the reaction mixture was stirred at room temperature for 3 hours. The unreacted tritium gas was re-absorbed to the manifold and the catalyst was removed through a Whatman Autovial (0.45 u PTFE) syringless filter. The solvent was removed under reduced pressure using rotary evaporator. The residual was subjected to removal of the beta-position tritium using a mixture of methanol (50 mL) and 0.2 N NaOH (4 mL) with vigorously stirring at room temperature for 30 minutes and rota-evaporated to dryness. This process was repeated three times and the final material was subjected to HPLC purification on Phenomenex Curasil column at 254 nm, eluted with 80% aqueous (0.1% TFA) and 20% acetonitrile isocratically in 30 min at 5 mL/min. The combined fractions of HPLC were concentrated by passing through a Sep-Pak C18 cartridge to afford 32 mCi of the title compound Phenyl-4-³H, pyrindine-4-³H in 50 mL ethanol (Specific activity=40.7 Ci/mmol).

### Example 4

### N-(4-{[(2-benzoylphenyl)amino]sulfonyl}phenyl)pyridine-2-carboxamide

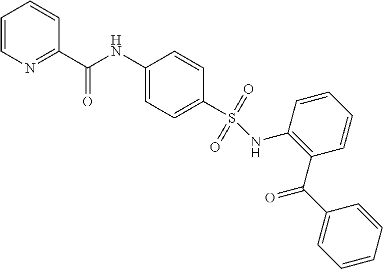

### Step 1: N-Phenylpyridine-2-carboxamide

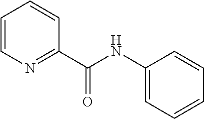

To a stirred solution of aniline (1.0 g, 10.7 mmol) in anhydrous THF (20 mL) was added triethylamine (4.5 mL, 32.4 mmol) and picolinoyl chloride hydrochloride (2.87 g, 16.2 mmol). The reaction mixture was stirred at r.t. for 2 h, diluted with EtOAc, and washed with brine and aqueous NaHCO₃. The aqueous layer was washed with EtOAc and the combined organic layers were dried over Na₂SO₄. The solution was then filtered through a pad of silica using EtOAc as the eluent, and the solvent was removed in vacuo to yield the title compound (1.95 g, 92%) as a solid. LRMS (ESI) m/z 199.3 [(M+H)⁺; calcd for C₁₂H₁₁N₂O: 199.1].

### Step 2: 4-[(pyridin-2-ylcarbonyl)amino]benzenesulfonyl chloride

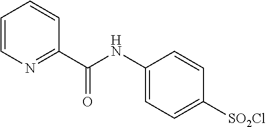

To N-phenylpyridine-2-carboxamide (500 mg, 2.5 mmol) was added chlorosulfonic acid (1.5 g, 12.8 mmol) and the mixture was stirred at r.t. for 1 h. The mixture was then poured into ice and stirred for 30 min. The resulting solids were then collected by filtration, washed with water, and air dried to yield the title compound (0.71 g, 96%) as a white solid. LRMS (ESI) m/z 297.2 [(M+H)⁺; calcd for C₁₂H₁₀ClN2O3S: 297.0].

### Step 3: N-(4-{[(2-benzoylphenyl)amino]sulfonyl}phenyl)pyridine-2-carboxamide

To (2-aminophenyl)(phenyl)methanone (17 mg, 0.086 mmol) and 4-[(pyridin-2-ylcarbonyl)amino]benzenesulfonyl chloride (25 mg, 0.085 mmol) was added pyridine (0.25 mL) and the mixture was stirred at r.t. for 8 h. The mixture was then diluted with 10% water/DMSO (0.6 mL) and purified by reverse phase chromatography (30-100% acetonitrile/0.015% TFA-water) to yield the title compound. (ESI) m/z 458.3 [(M+H)⁺; calcd for C₂₅H₂₀N₃O₄S: 458.1].

The following compounds in Table 1 were made by the general synthesis set out above, with appropriate modification to the reagents.

TABLE 1

| EXAMPLE | STRUCTURE | (M + H)⁺ |
|---|---|---|
| 5 | 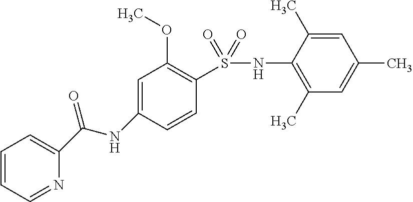 | 426 |
| 6 | 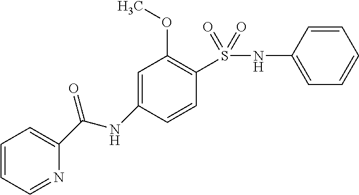 | 384 |
| 7 | 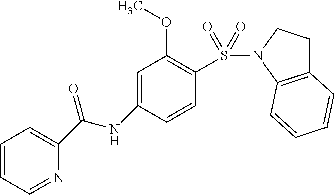 | 410 |
| 8 | 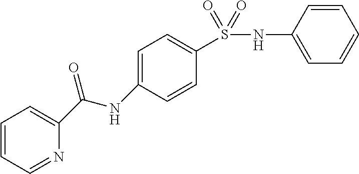 | 354 |
| 9 | 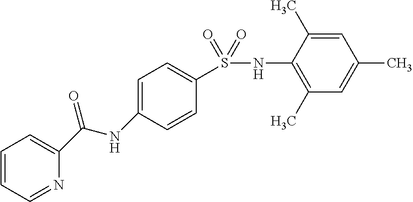 | 396 |
| 10 | 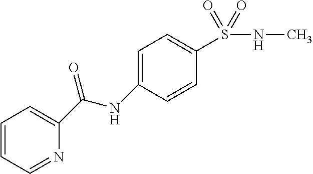 | 292 |
| 11 | 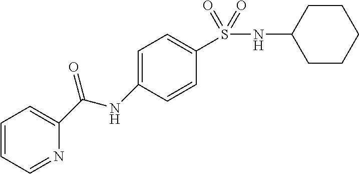 | 360 |
| 12 | 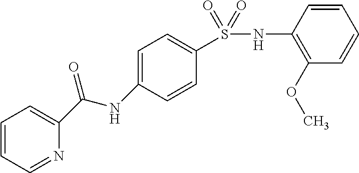 | 384 |
| 13 | 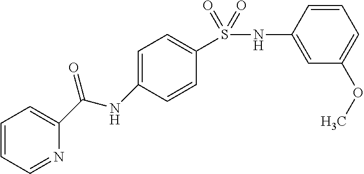 | 384 |
| 14 | 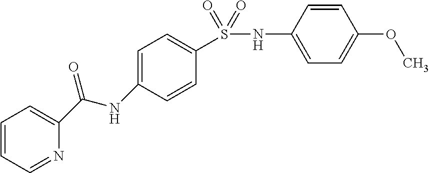 | 384 |
| 15 | 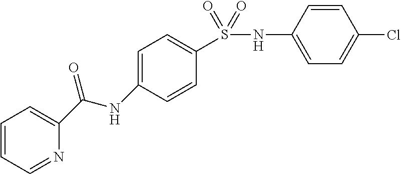 | 388 |
| 16 | 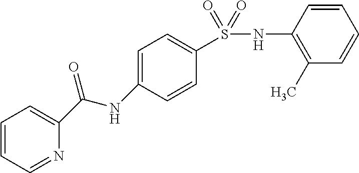 | 368 |
| 17 | 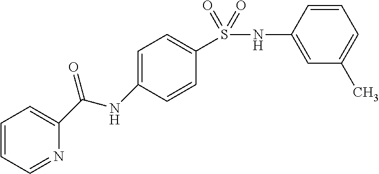 | 368 |
| 18 | 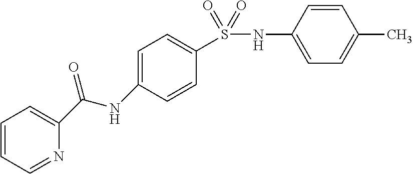 | 368 |
| 19 | 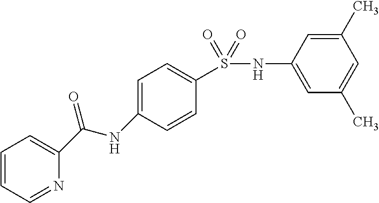 | 382 |
| 20 | 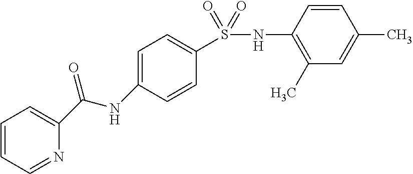 | 382 |
| 21 | 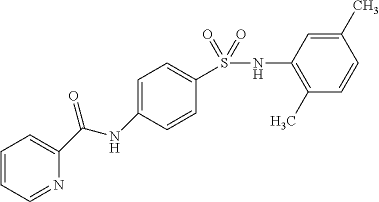 | 382 |
| 22 | 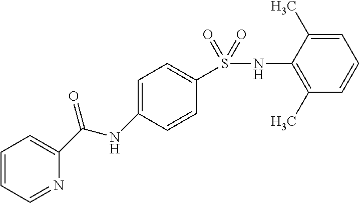 | 382 |
| 23 | 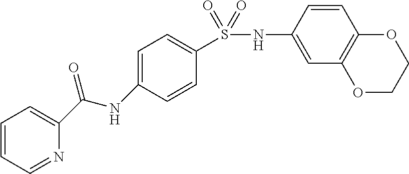 | 412 |
| 24 | 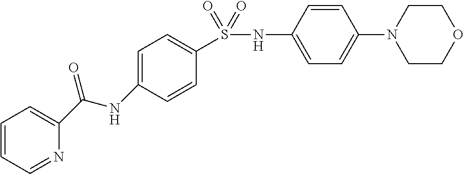 | 439 |
| 25 | 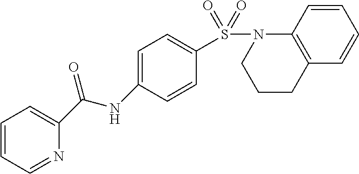 | 394 |
| 26 | 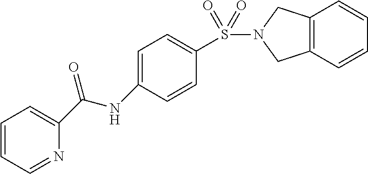 | 380 |
| 27 | 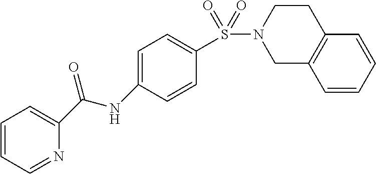 | 394 |
| 28 | 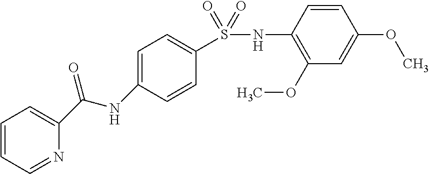 | 414 |
| 29 | 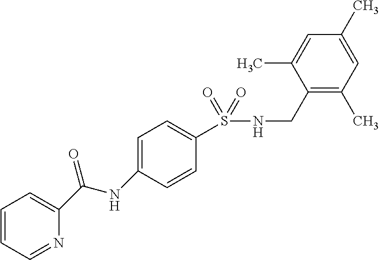 | 410 |
| 30 | 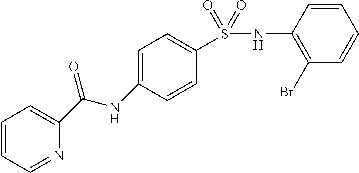 | 432 |
| 31 | 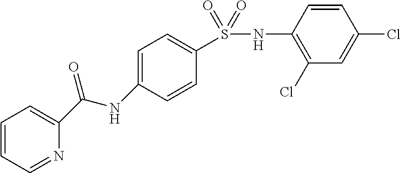 | 422 |
| 32 | 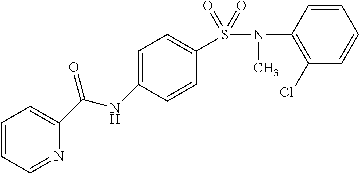 | 402 |
| 33 | 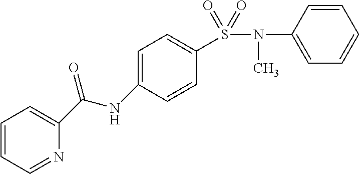 | 368 |
| 34 | 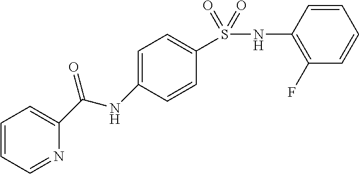 | 372 |
| 35 | 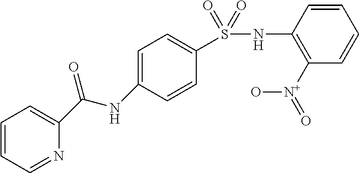 | 399 |
| 36 | 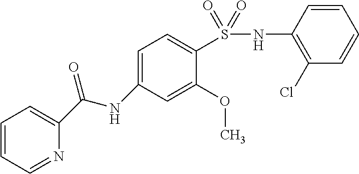 | 418 |
| 37 | 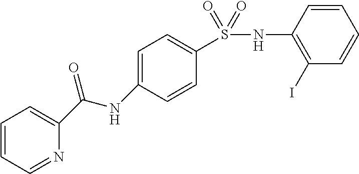 | 480 |
| 38 | 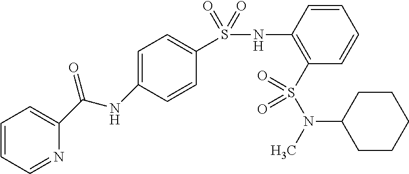 | 529 |
| 39 | 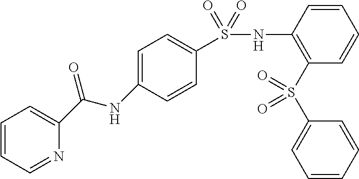 | 494 |
| 40 | 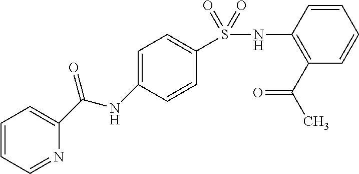 | 396 |
| 41 | 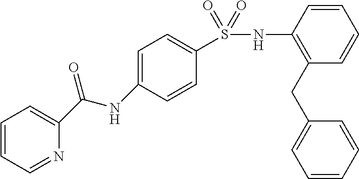 | 444 |
| 42 | 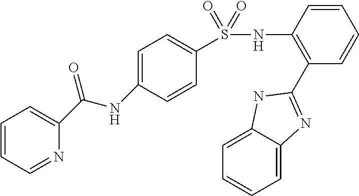 | 470 |
| 43 | 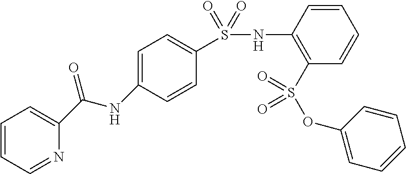 | 510 |
| 44 | 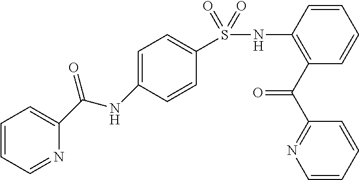 | 459 |
| 45 | 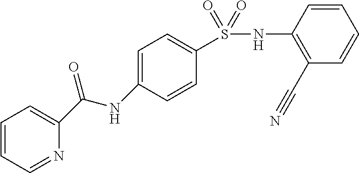 | 379 |
| 46 | 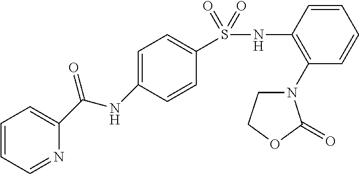 | 439 |
| 47 | 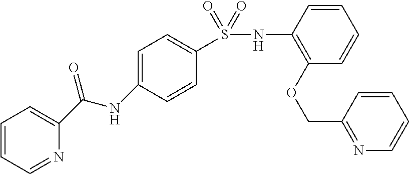 | 461 |
| 48 | 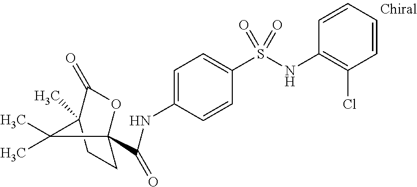 | 463 |
| 49 | 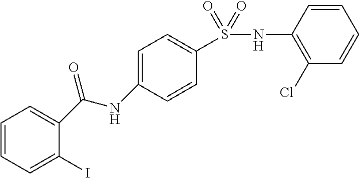 | 513 |
| 50 | 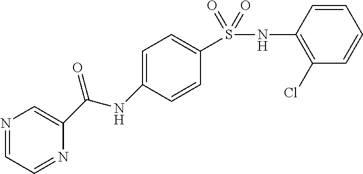 | 389 |

mGluR4 Binding Assay

Membranes were prepared from CHO cells expressing the human mGluR4 receptor. Cells were harvested using trypsin and then washed in assay buffer, assay buffer contains 50 mM HEPES, 1 mM EDTA and protease inhibitors (Boehringer Mannheim catalog #1 836 170). Harvested cells were pelleted by centrifugation and stored at −80° C. for a minimum of 12 hours. Cells were resuspended in assay buffer and lysed by a polytron, a low speed spin, 2000×g for 10 min, was used to recover unbroken cells for additional treatment using the polytron. The cleared supernatant from the low speed spins was subjected to a high speed spin, 96,000×g for 1 hour. The membrane pellet from the high speed spin was resuspended in assay buffer and used in the mGluR4 binding assay.

The binding assay follows a standard filtration binding paradigm. The binding buffer consists of 20 mM HEPES, 100 mM NaCl and 3 mM MgCl₂, the pH of the buffer is 7.4. The assay is performed in a 96-well format with each well containing test compound, 25 to 50 ug of membrane protein, 20 uM L-AP4, and 7 nM radiotracer, [³H]-N-[3-Chloro-4-(4-methyl-1,3-dioxo-1,3-dihydro-2H-isoindol-2-yl)phenyl]pyridine-2-carboxamide, which is disclosed in commonly owned U.S. patent application docket no. MRL-NOP-00030-US-PSP, filed contemporaneously with this application.

The binding reaction is incubated at room temperature for 1 hour, and then is passed through GF/B filter that was presoaked with 0.5% PEI. The cell membranes are collected on the filter and then washed five times with ˜5 ml ice cold binding buffer that contains 0.01% BSA. The washed filter plate is dried for 30 minutes at 37° C., scintillation fluid is added, and the amount of filter bound radioactivity is determined using a TopCount instrument. Total binding is determined in the absence of any test compound, but in the presence of the DMSO concentration that would result from the addition of test compound. To determine the level of non-specific binding for the radiotracer [³H]-N-[3-Chloro-4-(4-methyl-1,3-dioxo-1,3-dihydro-2H-isoindol-2-yl)phenyl]pyridine-2-carboxamide, 5 uM of unlabeled [³H]-N-[3-Chloro-4-(4-methyl-1,3-dioxo-1,3-dihydro-2H-isoindol-2-yl)phenyl]pyridine-2-carboxamide, is added to determine the level of non-specific binding.

A 10-point dose response, with either 2-fold or 3-fold dilution steps, is performed and curve fitting algorithms, suitable for a sigmoidal dose response relationship, are used to fit the data. The inflection point of the curve, or IC₅₀, provides a measure of the affinity of a test compound for the mGluR4 receptor.

Compounds of the invention, in general, have activity in the mGluR4 binding assay of IC50<10 uM. Representative values for the mGluR4 binding assay for the compounds of the invention are set forth in Table 2 below:

TABLE 2

| EXAMPLE | IC50 (nM) |
|---|---|
| 1 | 33 |
| 6 | 6,076 |
| 18 | 1,104 |
| 30 | 141 |
| 41 | 182 |

Fluorescent Imaging Plate Reader (FLIPR) Assay for mGluR4 Potentiators

Cells are grown in Corning T162 cm² flasks in DMEM growth media at 37° C. and 5% CO₂. Growth media contains: Gibco DMEM supplemented with 10% defined FBS, 1% GlutaMax −1, 1% sodium pyruvate, 1% NEAA, 1% Pen/Strep, 250 ug/ml zeocin, 25 mM HEPES, 0.1% 2-Mercaptoethanol, 600 ug/ml Hygromycin B, 7.5 ug/ml blasticidin, and 5 ug/ml puromycin.

The day prior to the experiment the cells were plated in growth medium (minus glutamine) at 50K cells/well into Becton-Dickinson 384-well PDL-coated plates using a Labsystems Multidrop (0.1 ml/well). The cells are grown overnight at 37° C. and 6% CO₂.

The following day, the cells are washed with Assay Buffer at 37° C. using a Skatron EMBLA cell washer (3×100 μl, aspiration 3 mm from bottom will leave 30 μl of buffer in each well). Assay buffer is Hanks balanced salt solution with 20 mM HEPES, 2.5 mM probenecid and 0.1% BSA.

Fluorescent dye, 30 μl of the diluted Fluo-4-AM, is added to each well of the plate. The Fluo-4-AM solution is added to achieve a final concentration in the assay of 2 uM Fluo-4-AM, 0.02% pluronic acid and 1% FBS. The assay plates are placed back into the 6% CO₂ incubator and incubated at 37° C. for 60 min. The 384-well plates are washed with Assay Buffer at 37° C. on using a cell washer (3×100 μl, aspiration 3 mm from bottom, will leave 30 μl of buffer in each well). The experimental run is initiated and after 30 sec a dose of test compound is applied. The fluorescent response elicited by test compound is monitored for 5 min, any positive response detected at this stage is attributed to agonist properties of the test compound. At this point an EC10 concentration of the mGluR4 agonist L-AP4 ((S)-2-amino-4-phosphonobutanoate) (final concentration 30 nM) is added and the fluorescent response is monitored for an additional 3 minutes, any positive response detected at this stage is attributed to potentiator properties of the test compound.

Controls for the assay include a no compound control, a positive control which is a saturating concentration, 10 uM, of the agonist L-AP4. A 10 point dose response, with either 2-fold or 3-fold dilution steps, is performed and curve fitting algorithms, suitable for a sigmoidal dose response relationship, are used to fit the data. The inflection point of the curve provides a measure of compound potency.

A comparison of the maximal level of activity achieved for a test compound, relative to the response achieved with a saturating concentration of the agonist L-AP4 provides a measure of compound efficacy.

Compounds of the invention, in general, have an mGluR4 FLIPR potency of <10 uM. Representative values for the mGluR4 FLIPR assay for the compounds of the invention are set forth in Table 3 below.

TABLE 3

| | mGluR4 FLIPR Potency | mGluR4 FLIPR % |
|---|---|---|
| EXAMPLE | (nM) | Max Efficacy |
| 1 | 162 | 111 |
| 6 | 3,940 | 70 |
| 18 | 1,350 | 93 |
| 30 | 134 | 110 |
| 41 | 480 | 124 |

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention.

What is claimed is:

1. A compound of formula (I): 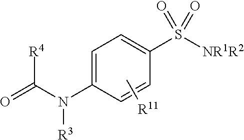
wherein:
R¹ and R² are each independently selected from the group consisting of
(1) hydrogen,
(2) —C₆₋₁₀ aryl,
(3) heteroaryl,
(4) —C₁₋₆ alkyl,
(5) —C₃₋₈ cycloalkyl,
wherein said R¹ or R² alkyl, cycloalkyl, aryl or heteroaryl moiety is optionally substituted with one or more
(a) halogen,
(b) —C₆₋₁₀ aryl,
(c) heteroaryl,
(d) —OC₁₋₄ alkyl,
(e) —C₁₋₄ alkyl,
—CN,
(g) —NO₂,
(h) —C(═O)—R⁵,
(i) —S(O)ₙR⁵,
(j) —S(O)ₙ—NR⁹R¹⁰,
(k) —S(O)ₙ—O—R⁵,
(l) —C(═O)—OR⁵,
(m) —NR⁹R¹⁰,
wherein said alkyl or aryl moiety is optionally substituted with one or more
(i) halogen,
(ii) —CN,
(iii) —OC₁₋₄ alkyl,
(iv) —C₁₋₄ alkyl,
(v) —C₆₋₁₀ aryl,
(vi) heteroaryl,
or R¹ and R² are linked together with the nitrogen to which they are both attached to form a monocyclic or bicyclic heterocyclic group, wherein said heterocyclic group is optionally substituted with one or more
(a) halogen,
(b) —C₁₋₄ alkyl, or
(c) —OC₁₋₄ alkyl, or
provided that R¹ and R² are not both hydrogen;
R³ is selected from the group consisting of
(1) hydrogen, or
(2) —C₁₋₄ alkyl;
R⁴ is a heteroaryl group having at least one nitrogen atom, selected from the group consisting of pyridine, pyrimidine and quinolinimide, wherein said R⁴ heteroaryl is optionally substituted with one or more
(1) —C(═O)—OR⁵,
(2) —C₁₋₄ alkyl,
(3) —OC₁₋₄ alkyl, or
(4) halogen,
wherein said alkyl moiety is optionally substituted with one or more
(a) halogen, or
(b) hydroxyl;
R⁵ is selected from the group consisting of
(1) hydrogen,
(2) —C₁₋₄ alkyl, or
(3) —C₆₋₁₀ aryl;
R⁶, R⁷ and R⁸ are each independently selected from the group consisting of
(1) hydrogen, or
(2) —C₁₋₄ alkyl;
R⁹ and R¹⁰ are each independently selected from the group consisting of
(1) hydrogen,
(2) —C₁₋₄ alkyl
(3) —C₆₋₁₀ aryl, or
(4) —C₃₋₈ cycloalkyl,
or R⁹ and R¹⁰ are linked together to form a heterocyclic group,
R¹¹ is present at one or more of the phenyl ring carbon atoms, and each R¹¹ is independently selected from the group consisting of
(1) —C(═O)—OR⁵,
(2) —C₁₋₄ alkyl,
(3) —OC₁₋₄ alkyl, or
(4) halogen,
wherein said alkyl moiety is optionally substituted with one or more
(a) halogen, or
(b) hydroxyl;
n is 0, 1 or 2;
or a radiolabeled derivative thereof, and pharmaceutically acceptable salts thereof.

2. A compound of claim 1, or a pharmaceutically acceptable salt thereof, wherein R¹ is hydrogen or methyl and R² is selected from the group consisting of
(1) —C₆₋₁₀ aryl,
(2) heteroaryl,
(3) —C₁₋₆ alkyl,
(4) —C₃₋₈ cycloalkyl,
wherein said R² alkyl, cycloalkyl, aryl or heteroaryl moiety is optionally substituted with one or more
(a) halogen,
(b) —C₆₋₁₀ aryl,
(c) heteroaryl,
(d) —OC₁₋₄ alkyl,
(e) —C₁₋₄ alkyl,
(f) —CN,
(g) —NO₂,
(h) —C(═O)—R⁵,
(i) —S(O)ₙR⁵,
(j) —S(O)ₙ—NR⁹R¹⁰,
(k) —S(O)ₙ—O—R⁵,
(l) —C(═O)—OR⁵,
(m) —NR⁹R¹⁰,
wherein said alkyl or aryl moiety is optionally substituted with one or more
(i) halogen,
(ii) —CN,
(iii) —OC₁₋₄ alkyl,
(iv) —C₁₋₄ alkyl,
(v) —C₆₋₁₀ aryl, or
(vi) heteroaryl.

3. A compound of claim 2, or a pharmaceutically acceptable salt thereof, wherein R¹ is hydrogen or methyl and R² is selected from the group consisting of
(1) phenyl,
(2) pyridyl,
(3) pyrimidyl, or
(4) —C₁₋₆ alkyl, optionally substituted
wherein said R² is optionally substituted with one or more
(a) halogen,
(b) —OC₁₋₄ alkyl,
(c) —C₁₋₄ alkyl,
(d) phenyl,
(d) —CN,
(e) —C(═O)—R⁵,
(f) —C(═O)—OR⁵, or
(g) —NR⁹R¹⁰.

4. A compound of any of claims 1 to 3, or a pharmaceutically acceptable salt thereof, wherein R³ is hydrogen.

5. A compound of any of claims 1 to 4, or a pharmaceutically acceptable salt thereof, wherein R⁴ is pyridine.

6. A compound of claim 1, which is a compound of formula (II): 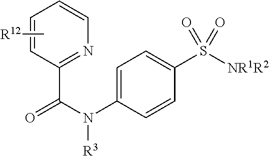
or a radiolabeled derivative thereof, and pharmaceutically acceptable salts thereof, wherein R¹, R² and R³ are as defined in claim 1, and R¹² is present at one or more of the pyridine ring carbon atoms, and each R¹² is independently selected from the group consisting of
(1) —C(═O)—OR⁵,
(2) —C₁₋₄ alkyl,
(3) —OC₁₋₄ alkyl, or
(4) halogen,
wherein said alkyl moiety is optionally substituted with one or more
(a) halogen, or
(b) hydroxyl.

7. A compound of claim 6, wherein R³ is hydrogen.

8. A compound of claim 1, which is a compound of formula (III): 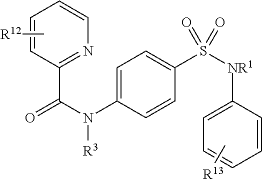
or a radiolabeled derivative thereof, and pharmaceutically acceptable salts thereof, wherein R¹ and R³ are as defined in claim 1, R¹² is present at one or more of the pyridine ring carbon atoms, and each R¹³ is independently selected from the group consisting of
(1) —C₆₋₁₀ aryl,
(2) heteroaryl,
(3) —C₁₋₆ alkyl,
(4) —C₃₋₈ cycloalkyl,
wherein said R² alkyl, cycloalkyl, aryl or heteroaryl moiety is optionally substituted with one or more
(a) halogen,
(b) —C₆₋₁₀ aryl,
(c) heteroaryl,
(d) —OC₁₋₄ alkyl,
(e) —C₁₋₄ alkyl,
(f) —CN,
(g) —NO₂,
(h) —C(═O)—R⁵,
(i) —S(O)₆R⁵,
(j) —S(O)ₙ—NR⁹R¹⁰,
(k) —S(O)ₙ—O—R⁵,
(l) —C(═O)—OR⁵,
(m) —NR⁹R¹⁰,
wherein said alkyl or aryl moiety is optionally substituted with one or more
(i) halogen,
(ii) —CN,
(iii) —OC₁₋₄ alkyl,
(iv) —C₁₋₄ alkyl,
(v) —C₆₋₁₀ aryl, or
(vi) heteroaryl.

9. A compound of claim 8, or a pharmaceutically acceptable salt thereof, wherein R³ is hydrogen.

10. A compound of claim 1, which is selected from the group consisting of
N-(4-{[(2-Chlorophenyl)amino]sulfonyl}phenyl)pyridine-2-carboxamide;
N-(4-{[(2-Chloro-4-iodophenyl)amino]sulfonyl}phenyl)-4-iodopyridine-2-carboxamide;
[³H]-N-(4-{[(2-Chlorophenyl)amino]sulfonyl}phenyl)pyridine-2-carboxamide;
N-(4-{[(2-benzoylphenyl)amino]sulfonyl}phenyl)pyridine-2-carboxamide
or a pharmaceutically acceptable salt thereof.

11. A compound of claim 1, which is selected from the group consisting of 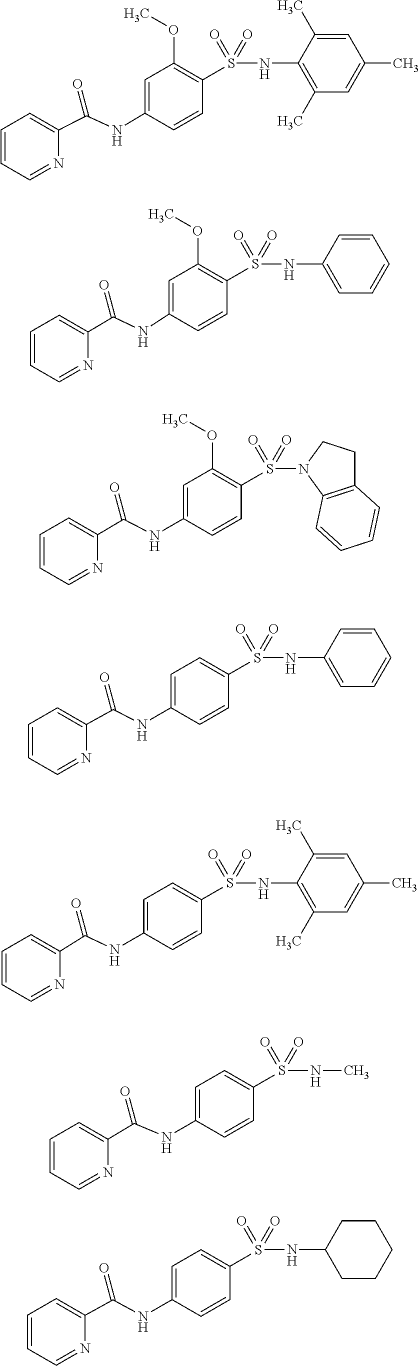 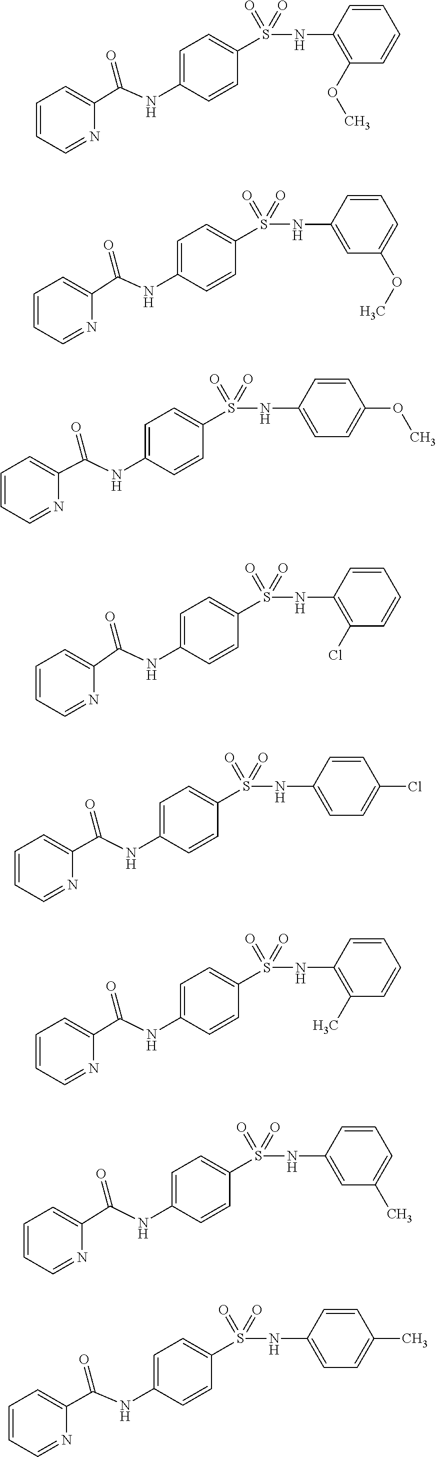 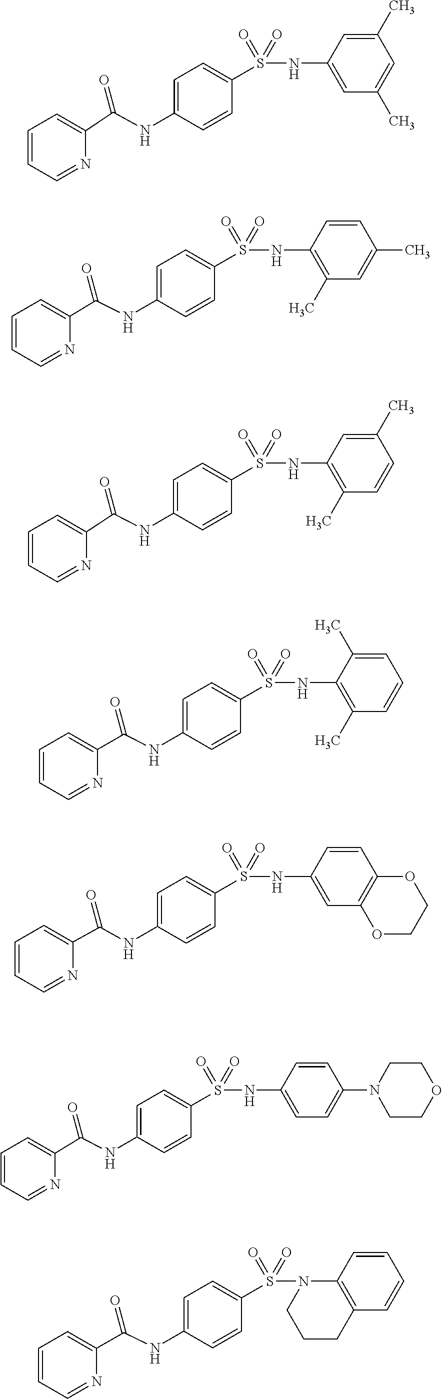 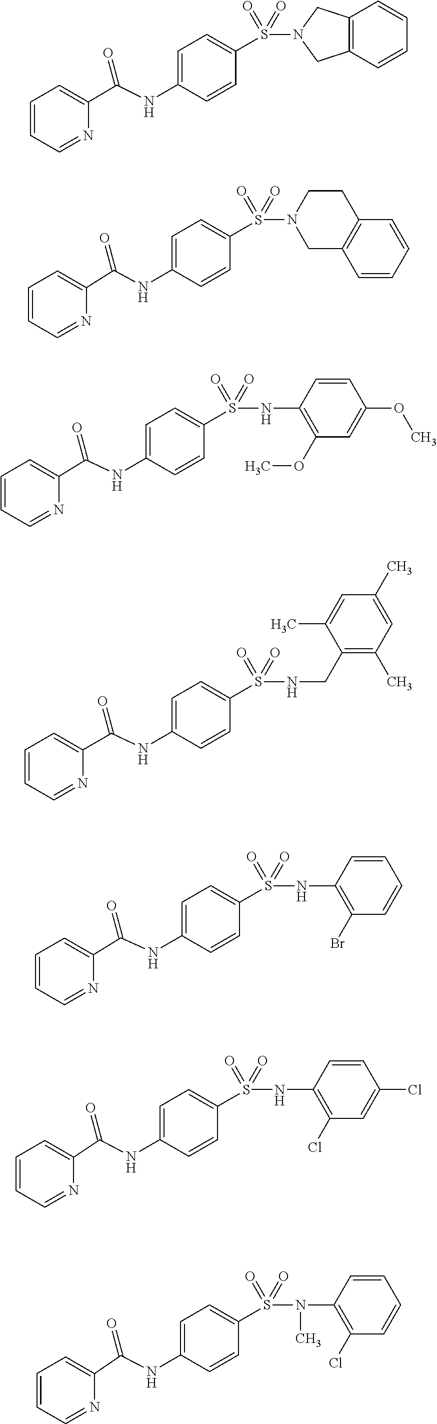 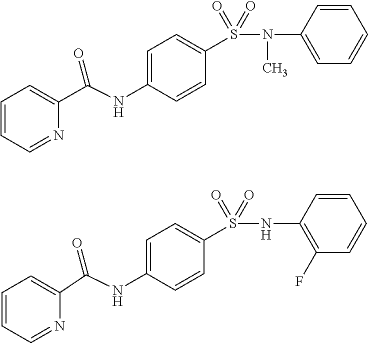 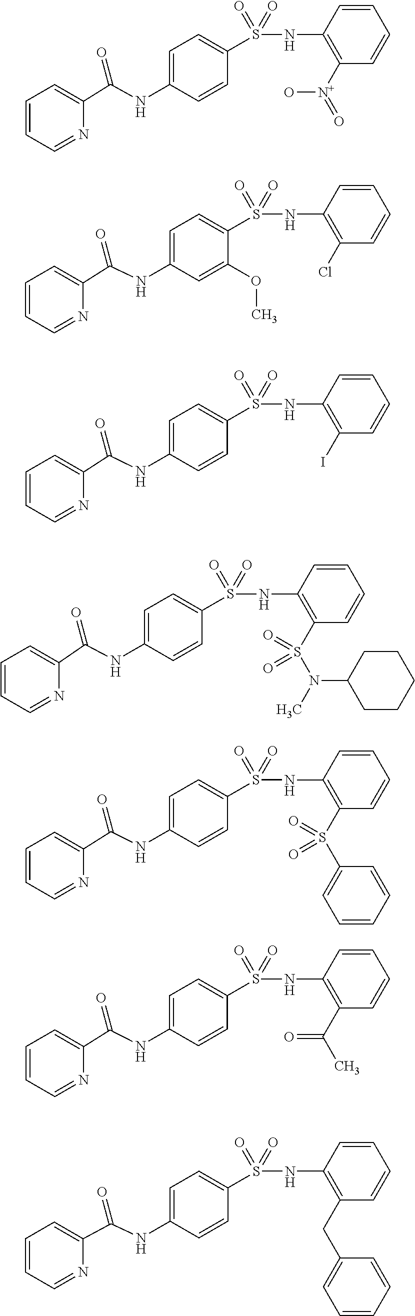 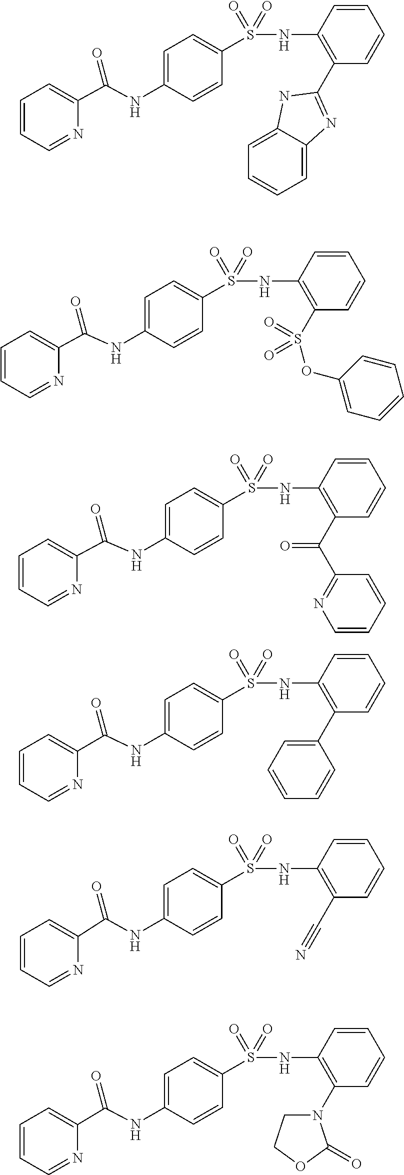 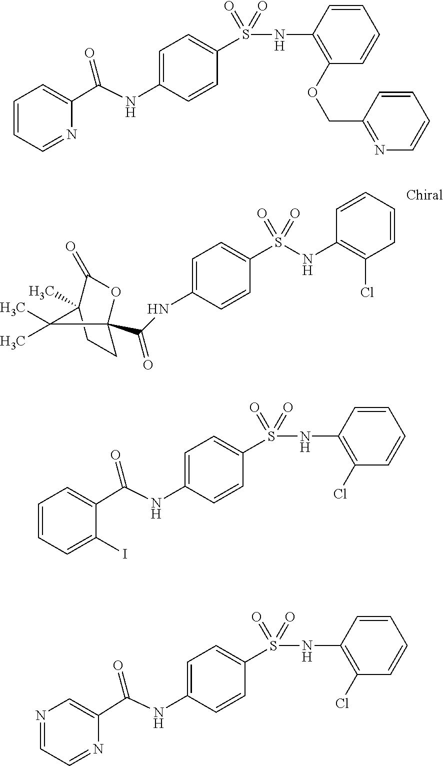
or a radiolabeled derivative, and pharmaceutically acceptable salts thereof.

12. A compound of claim 1, which is 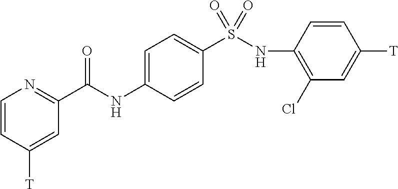
or a pharmaceutically acceptable salt thereof.

13. A radiopharmaceutical composition which comprises a radiolabeled compound of claim 1 and a pharmaceutically acceptable carrier or excipient.

14. A method for the manufacture of a medicament for the diagnostic imaging of mGluR4 in a mammal, which comprises combining the compound of claim 1 or a pharmaceutically acceptable salt thereof with a pharmaceutically acceptable carrier or excipient.

15. A method for the diagnostic imaging of mGluR4 in a mammal which comprises administering to the mammal in need of diagnostic imaging an effective amount of the compound of claim 1, or a pharmaceutically acceptable salt thereof, and obtaining an image of mGluR4 in the mammal using positron emission tomography.

16. A method for the diagnostic imaging of the brain in a mammal which comprises administering to a mammal in need of such diagnostic imaging an effective amount of the compound of claim 1, or a pharmaceutically acceptable salt thereof, and obtaining an image of the brain in the human using positron emission tomography.

17. A method for the diagnostic imaging of a disease or disorder in a human which comprises administering to the mammal in need of such diagnostic imaging an effective amount of a compound of claim 1, or a pharmaceutically acceptable salt thereof, and obtaining an image of the human using positron emission tomography.

18. A method for the diagnostic imaging of tissues bearing mGluR4 in a mammal which comprises administering to a mammal in need of such diagnostic imaging an effective amount of the compound of claim 1, or a pharmaceutically acceptable salt thereof, and obtaining an image of the tissues using positron emission tomography.

19. A method for the quantification of mGluR4 in mammalian tissue which comprises contacting such mammal tissue in which such quantification is desired with an effective amount of the compound of claim 1, or a pharmaceutically acceptable salt thereof, and detecting or quantifying the mGluR4 using positron emission tomography.

20. An assay for determining the binding affinity of a test compound to the mGluR4 receptor, comprising the steps of
(1) preparing a membrane from a cell expressing the human mGluR4 receptor;
(2) forming a solution comprising
(a) the membrane,
(b) a radiolabeled compound of claim 1,
(c) a test compound, and
(d) an mGluR4 orthosteric agonist;
(3) incubating the solution;
(4) collecting the membrane from the solution;
(5) determining the amount of radioactivity bound to the mGluR4 receptor; and
(6) calculating the affinity of the test compound for the mGluR4 receptor.

21. The method of claim 20, wherein the compound of claim 1 is radiolabeled with tritium.

22. The method of claim 20, wherein the mGluR4 orthosteric agonist is L-AP4.

23. The method of claim 20, wherein the compound of claim 1 is N-(4-{[(2-Chlorophenyl)amino]sulfonyl}phenyl)pyridine-2-carboxamide, radiolabeled with tritium.

24. The method of claim 23, wherein the compound of claim 1 is [³H]-N-(4-{[(2-Chlorophenyl)amino]sulfonyl}phenyl)pyridine-2-carboxamide [chemical structure]
